(19) United States
(12) Patent Application Publication
(10) Pub. No.: US 2007/0276029 A1
(43) Pub. Date: Nov. 29, 2007

(54) Glutamine-Containing Energy Imparting Amino Acid Composition or Amino Acid Solution

(76) Inventors: Takashi Abe, Saitama (JP); Reiko Inaba, Saitama (JP); Yutaka Fujiwara, Gunma (JP)

Correspondence Address: YOUNG & THOMPSON, 745 SOUTH 23RD STREET, 2ND FLOOR, ARLINGTON, VA, 22202, US

(21) Appl. No.: 10/583,037

(22) PCT Filed: Dec. 16, 2004
(86) PCT No.: PCT/JP04/18784

§ 371 Date: Jun. 11, 2007

(30) Foreign Application Priority Data
Dec. 16, 2003 (JP) 2003-417463

(51) Int. Cl. A61K 31/40; A61P 3/00

(52) U.S. Cl. 514/428

(57) ABSTRACT

[Problems] Amino acid composition with substantially no side effects which can not only efficiently supplement energy but also elevate the motor function. [Means for Solving Problems] Energy-imparting amino acid composition or amino acid solution containing glutamine comprising proline, alanine, valine, isoleucine, lysine and the glutamine. The energy-imparting amino acid composition is superior to the conventional amino acid compositions containing no glutamine in the energy supplementation and the motor function.

### TECHNICAL FIELD

The present invention relates to an energy-imparting amino acid composition or amino acid solution containing glutamine which implements elevation of motor functions (restoration from fatigue) in addition to energy complement.

### BACKGROUND ART

The present inventors have investigated saliva secreted from the larva of a hornet, and have examined the use thereof together with clarification of the formulation of the amino acid composition contained therein.

As a result, the amino acid composition named with VAAM among a plenty -of the amino acid compositions contained in the above saliva has been found to have a function of accentuating motor functions (Japanese patent No. 2518692). The functions of accentuating the motor functions include muscle endurance, alimentation and tonicity, and fatigue restoration. The VAAM dissolved in water is available in the market as isotonic drink.

A variety of isotonic drinks other than the VAAM are known which are prepared by dissolving compositions of various amino acids into water. The various types of the isotonic drinks are known with respect to their functions, which include a type of supplementing the nutrients lost during exercise and another type of generating the energy by burning internal fats such as the VAAM.

Various essential amino acids are used for forming the above isotonic drink compositions. However, glutamine, one of the amino acids, is not used for the component of the isotonic drinks (water soluble). This is because the glutamine exists in a human body relatively abundantly, and the glutamine is oxidatively decomposed into glutamic acid in many cases when it is dissolved into a solution to become instable.

Almost all the glutamine is ordinarily synthesized in the human body and the intake thereof through a meal is seldom required. When, however, the body once sustains an invasion such as heat burn, external injury and medical operation, an amount of the consumed glutamine rapidly increases to bring about a relatively-deficiency state of glutamine. As described, the glutamine is supposed to promote its availability for the raw material of synthesizing a protein, the energy source for a cell such as an immune cell and various materials for restoring a wound or destroyed tissue and cell. Accordingly, the glutamine in the form of solution is desirably ingested against the rapid decrease of the glutamine in the human body.

However, as mentioned, it is the common knowledge among biochemists that even when the glutamine is used as the amino acid composition for the isotonic drinks, it is decomposed during the transportation and the preservation not to act as an effective component of the isotonic drinks. Accordingly, conventional isotonic drinks including the VAAM do not contain the glutamine as the component of the isotonic drinks.

For example, the glutamine is known to be available as a component of fluid infusion (JP-A-11(1999)-802164, claim 1). Also in this case, the glutamine is provided as a freeze-dry article or a is solid component which can be dissolved before its use,

### DISCLOSURE OF THE INVENTION

Problems to be Solved by the Invention

The glutamine supposed to be desirable as the component of the isotonic drinks is not used as the component due to the instability thereof in the solution.

Even if, as described, the glutamine is used as the component of the fluid infusion or as the freeze-dry article or the solid component which is dissolved before its use, we mainly purchase the isotonic drinks in a drugstore or a convenience store or from a vending machine. Accordingly, the glutamine is not supposed to be the component of the isotonic drinks or the fluid infusion which is maintained for a relatively longer period of time in a solution

The main function of the conventional isotonic drinks is supposed to supplement the nutrients lost in exercise, and the motor function is maintained by the supplement. If the motor functions of the human body can be elevated in addition that the nutrients lost in the exercise is simply supplemented by the amino acid, such amino acid composition can elevate the motor function in addition to having the function of the isotonic drinks.

Accordingly, an object of the present invention is to provide amino acid composition or amino acid solution which enables the elevation of the motor function of the human body other than having the advantages of isotonic drinks for supplementing energy.

Means for Solving Problems

The present invention provides, firstly, energy-imparting amino acid composition or amino acid solution containing glutamine comprising proline, alamine, valime, isoleucine, lysine and the glutamine (hereinafter referred to as first invention), secondly, energy-imparting amino acid composition or amino acid solution containing glutamine comprising proline, alanine, valine, isoleucine, lysine, the glutamine and citric acid (hereinafter referred to as second invention) and, thirdly, energy-imparting amino acid composition or amino acid solution containing glutamine comprising a plurality of essential amino acids, the glutamine and a glutamine-stabilizing sugar (hereinafter referred to as third invention).

The present invention will be described in detail

The present invention is characterized by the addition of glutamine which has not been practically contained in conventional amino acid composition or amino acid solution.

In the first and the second inventions, the glutamine is added to the conventional amino acid composition or amino acid solution containing proline, alanine, valine, isoleucine and lysine. The obtained amino acid composition or amino acid solution has a stronger function of elevating the motor function than the conventional one containing no glutamine. When, based on this knowledge, energy is loaded for obtaining the further elevated function by adding an organic acid such as citric acid, malic acid and fumaric acid, it appears apparent that the citric acid has the strongest loading effect. The addition of an excitometabolic agent such as vitamins and CoQ (coenzyme Q) 10 provides the amino acid composition having the further strong function of elevating the motor function. The respective amino acids are preferably L-amino acids.

While the instable glutasine is contained in the first and the second inventions as described above, the instability of the glutamine is not problematic in case that powdery mixture composition is used or the mixture is ingested immediately after its dissolution.

The decomposition of the glutamine must be prevented when the amino acid solution prepared by dissolving the composition containing the glutamine is preserved. The present inventors have conducted various investigations to find out a glutamine-stabilizing sugar which stably maintains the glutamine even in a solution.

The glutamine-stabilizing sugar includes trehalose.

Lactic acid is accumulated in blood as a result of fatigue due to continuous exercise. It is recognized that higher blood lactic acid concentration (or blood lactic acid value) indicates stronger fatigue, and adversely lower concentration indicates restoration from the fatigue. It is recognized, on the other hand, that higher glucose concentration (blood sugar level) indicates a large amount of energy is usable so that the motor function is maintained at a higher level to sufficiently supplement the energy. Further, it is recognized that a higher free fatty acid value maintains a higher motor function.

As described earlier, the amino acid composition or amino acid solution of the first invention contains, as the essential components, the six amino acids, that is, the proline, the alanine, the valine, the isoleucine, the lysine and the gluta-mine, and the amino acid composition or amino acid solution of the second invention contains the citric acid other than the six amino acids as the essential components. The respective amino acids are desirably contained in their specific composition ratios such as 4 to 30 moles of the proline, 0.1 to 12 moles of the alanine, 4 to 8 moles of the valine, 3 to -9 moles of the isoleucine, 5 to 11 moles of the lysine and 0.1 to 4 moles of the glutamine. In the second invention, 5 to 50 moles of the citric acid is further added. Amino acids other than the above, watersoluble vitamins, acids or a small amount of other additives may be contained.

In the third invention different from the first and the second inventions, the amino acids other than the glutamine are not restricted. The amino acid composition or amino acid solution containing a plurality of amino acids added of the glutamine and the glutamine-stabilizing sugar has the higher function of elevating the motor function than the corresponding amino acid composition containing no glutamine.

The amino acid composition of the first to the third inventions may be ingested as powder or solution after dissolution into water. The ingestion can be conducted through an ordinary administration method such as oral administration, rectum administration, injection and infusion administration

In case of the oral administration, other than the administration of the composition itself, the composition can be used as powder medicine, a granulated agent, a tablet, a capsule or a troche, together with the medically permissible support, shaping agent or diluting agent. However, a longer period of time may be required to absorb the solid powder medicine and the tablet so that the oral administration of the composition itself is preferable. In this case, the administration as a solution with a suitable additive such as a salt including sodium chloride, a pH adjuster or a chelating agent can be used. After the addition of a suitable buffer or isotonic agent and dissolution into sterilized and distilled water, the composition may be used as an injection agent.

The timing of ingesting the composition is not especially restricted, and the composition may be ingested at arbitrary timing before or after generation of central nervous fatigue. Especially, the intake as drinkable preparation (for example, cold beverage, powdery drinkable beverage, drinks as a medicine for a purpose of alimentation and tonicity and nutrition supplement) is preferable.

The composition of the present invention is extremely lowly toxic, and an amount of administration can be established in a significantly wider range. An amount of administration depends on an administration method and a target of use, and ordinarily, one dose is from 0.5 to 5 g and is preferably from 1 to 2 g, and daily dose is from 1 to 20 g, and preferably is from 4 to 10 g. When administrated or ingested as solution, the composition in about from 0.5 to 10% in weight of solution may be administrated or ingested by from 10 to 1000 ml per every dose, and preferably the composition in from 1 to 4% in weight of solution may be administrated or ingested by from 100 to 400 ml per every dose.

Effect of the Invention

When the amino acid compositions of the first and the second inventions and the conventional amino acid mixture “V9” which is recognized to exhibit the highest free fatty acid value during exercise are compared with one another comprehensively in connection with the free fatty acid value, the blood sugar value and the lactic acid value, the former two inventions are recognized to exhibit the more excellent characteristics and to maintain the higher motor function. A similar effect can be expected in the third invention which is prepared by adding the glutamine to the conventional amino acid composition or amino acid solution.

In spite of the recognition that the instability of the glutamine adversely affects the above characteristics so that the added glutamine is instable and cannot be the component of the isotonic drinks, the glutamine-stabilizing sugar such as trehalsoe may be added, thereby the practicable amino acid composition or amino acid solution can be readily provided. In case of the solution, it is desirably taken as soon as after the dissolution.

### BEST MODE FOR IMPLEMENTING THE INVENTION

Although Examples of the energy-imparting amino acid composition of the present invention will be described, the present invention shall not be-restricted thereto.

The blood sugar level in blood, the lactic acid value and the free fatty acid value used in the Examples are measured as follows.

1. Lactic Acid Level in Blood

The lactic acid in blood is a material generated by fatigue in exercise. The lactic acid level in blood was calculated by measuring the absorption of NADH at 280 nm. The NADH was produced by using a supernatant prepared by conducting the deproteinization on withdrawn blood with use of 6%-PCA (pyrrolidone carboxylic acid) in accordance with the following Lactate Dehydrogenase method.
Lactate+NAD→(Lactate Dehydrogenase)→Pyruvate+NADH
2. Blood Sugar Level (Amount of Glucose)

The blood sugar level was calculated by measuring the optical density (OD) of NADPH at 280 nm. The NADPH was produced by using, similarly to that of the lactic acid value in blood, a supernatant prepared in accordance with the following Hexokinase method.
D-glucose+ATP→(Hexokinase)→D-glucose-6-P+ADP
D-glucose-6-P+NADP→(G6P-Dehydrogenase)→D-gluconate-6-P+NADPH+H
3. Free Fatty Acid Value in Blood

The free fatty acid value was measured by using a supernatant (serum) prepared by, standing withdrawn blood for 30 minutes followed by centrifuging at 3000 rpm in accordance with the Enzyme method illustrated by the following formulae. In the formulae, “ACS”, “ACOD” and “PCO” are abbreviations of “Acyl-CoA Synthetase”, “Acyl-CoA Oxidase” and “Peroxidase”, respectively.

### EXAMPLE 1 AND COMPARATIVE EXAMPLE 1

### Exercise Test

Ddy-type male mice (SPF) (five to eight weeks from birth) were used. After no food was supplied to the mice for 16 hours, amino acid nutrient liquid (composition of V10 in Table 1, Example 1) was orally administered at a rate of 37.5 μl/g-body weight. Thereafter, weights of 0.3 g were attached to the mouse tails, and they were made swim with the loads in a river pool at 35° C. for 30 minutes. After the swimming, the blood sugar level, the lactic acid amount and the free fatty acid amount in the withdrawn blood were measured.

Further, the blood sugar level, the lactic acid amount and the free fatty acid amount in the blood were measured by using the same ddy-type -male mice (SPF) (five to eight weeks from birth) and amino acid nutrient liquid V9 in Table 1 (Comparative Example 1) under the same conditions.

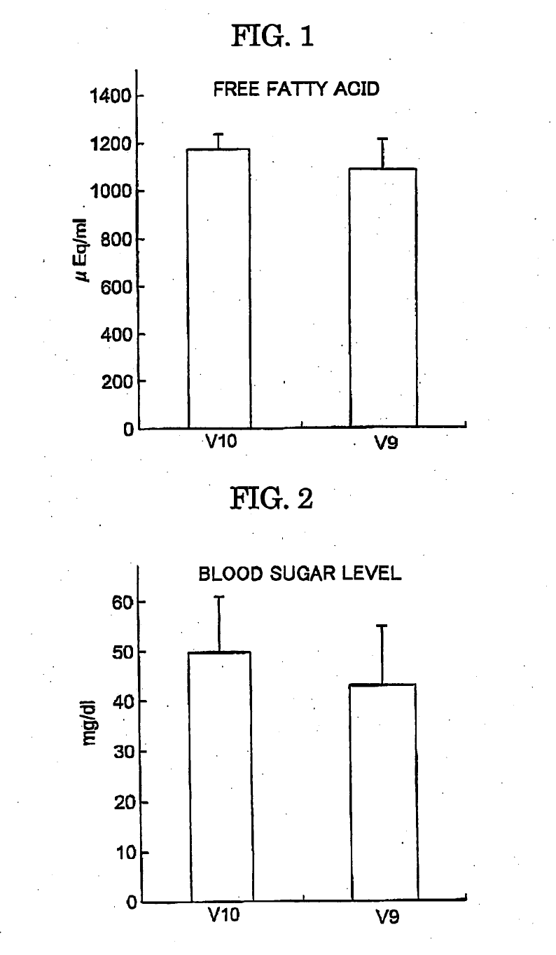

The free fatty acid amounts, the blood sugar levels and the lactic acid amounts of Example 1 and Comparative Example 1 were sequentially shown in graphs of FIGS. 1, 2 and 3.

When the amino acid mixture “V9” of Comparative Example 1 which has heretofore exhibited the highest free fatty acid value in blood during exercise was compared with the “V10” of Example 1 which was prepared by adding the glutamine to the “V9”, “V10” exhibited the higher value than “V9” as shown in the graph of FIG. 1. As shown in FIG. 2, the blood sugar level of “V10” of the Example 1 was also higher, and inversely the lactic acid value of “V10”, of Example 1 was lower.

These results exhibit that “V10” of Example 1 has the higher function of elevating the motor functions than “V9” of Comparative Example 1 in a comprehensive manner.

### EXAMPLE 2

The free fatty acid amounts, the blood sugar levels and the lactic acid value in the blood of the mice were measured under the same conditions of Example 1 by using (1) “V10” of Example 1, (2) o acid nutrient liquid prepared by adding 3%-citric acid to “V10” and (3) amino acid nutrient liquid prepared by adding 3%-citric acid and 0.1%-CoQ 10 to “V10”.

These values were sequentially shown in FIGS. 4, 5 and 6.

As apparent from the respective graphs, in the amino acid nutrient liquid prepared by adding 3%-citric acid to “V10”, the free fatty acid amount decreased, and though the lactic acid value slightly increased, the blood sugar level increased.

Further, in the amino acid nutrient liquid prepared by adding 3%-citric acid and 0.1%-CoQ10 to “V10”, the decrease of the free fatty acid amount was similar to that of “V10+3%-citric acid” which was larger than that of “V10”. The blood sugar value was slightly higher than those of “V10” itself and “V10+3%-citric acid”, and the lactic acid value was slightly lower than those of “V10” itself and “V10+3%-citric acid”.

These results have revealed that the characteristics of the amino acid nutrient liquid can be adjusted by the addition of citric acid or CoQ10 to “V10”. Accordingly, the determination of the presence, the absence and its amount of the additives depending on the characteristics required in isotonic drinks can provide the isotonic drinks having the desired characteristics.

### EXAMPLE 3

The free fatty acid amounts, the blood sugar levels and the lactic acid values in the blood of the mice were measured under the same conditions of Example 1 by using (1) the liquid of amino acid nutrient prepared by adding 3%-citric acid to “V10” in Example 1, (2) the liquid of amino acid nutrient prepared by adding 3%-citric acid and 0.1% VM-RD-V having composition shown in Table 2 to “V10”, (3) the liquid of amino acid nutrient prepared by adding 8%-citric acid and 0.1% VM-RD2001 having composition shown in Table 3 to “V10”, and (4) the liquid of amino acid nutrient prepared by adding 3%-citric acid and 0.1% VM-Aqua 7 having composition shown in Table 4 to “V10”.

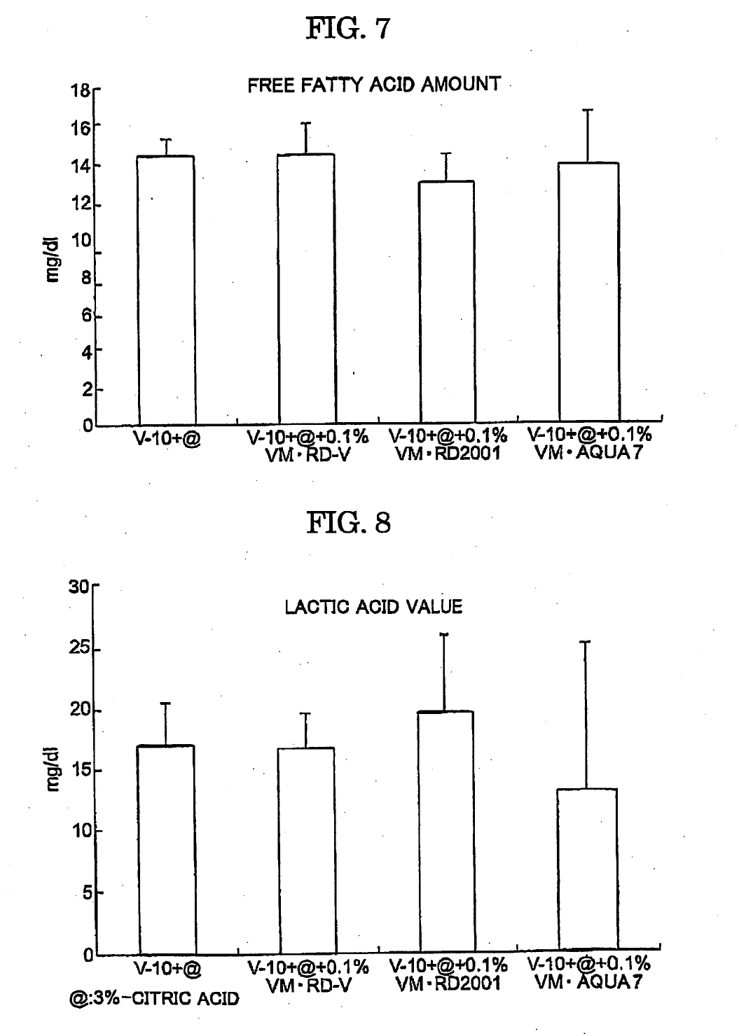

These values were sequentially shown in the graphs of FIGS. 7, 8 and 9.

As apparent from the respective graphs, among the vitamin mixtures, RD-V can suppress the degradation caused by the exercise in connection with the free fatty acid amount, the lactic acid value in blood and the blood sugar level more efficiently than RD2001 and the Aqua 7.

TABLE 1

| | | Comparative |
|---|---|---|
| Amino Acid | Example 1 (V10) | Example 1 (V9) |
| Proline | 37.5 molar % | 41.2 molar % |
| Alanine | 12.0 molar % | 13.2 molar % |
| Valine | 11.5 molar % | 12.6 molar % |
| Isoleucine | 8.9 molar % | 9.8 molar % |
| Lysine | 21.1 molar % | 23.2 molar % |
| Glutamine | 9.0 molar % | — |

TABLE 2
RD-V

| | | |
|---|---|---|
| Vitamin A | 16,650 | IU/g |
| Vitamin D₁ | 1,000 | IU/g |
| Extracted tocoferol | 50.0 | mg/g |
| Dibenzoyl thiamine hydrochloride (as thiamine | 50.0 | mg/g |
| hydrochloride) | (7.5 | mg/g) |
| Pyridoxine hydrochloride | 12.2 | mg/g |
| Cyanocobalamine | 10.0 | μg/g |
| Nicotinic acid amide | 95.0 | mg/g |
| Calcium pantothenate | 38.2 | mg/g |
| Folic acid | 1.0 | mg/g |
| L-ascorbic acid | 300.0 | mg/g |

TABLE 3
Aqua 7

| | | |
|---|---|---|
| Dibenzoyl thiamine hydrochloride (as thiamine | 12.9 | mg/g |
| hydrochloride) | (7.5 | mg/g) |
| Sodium riboflavin phosphate ester (as riboflavin) | 10.8 | mg/g |
| | (8.5 | mg/g) |
| Pyridoxine hydrochloride | 11.0 | mg/g |
| Cyanocobalamine | 30.0 | μg/g |
| Nicotinic acid amide | 65.0 | mg/g |
| Calcium pantothenate (as calcium) | 38.5 | mg/g |
| | (35.2 | mg/g) |
| L-ascorbic acid | 300.0 | mg/g |

TABLE 4
RD-2001

| | | |
|---|---|---|
| Vitamin A | 2,000 IU/300 | mg |
| Vitamin D | 100 IU/300 | mg |
| Vitamin E | 10 mg α -TE/300 | mg |
| Vitamin B1 | 1.1 mg/300 | mg |
| Vitamin B2 | 1.2 mg/300 | mg |
| Niacin | 17 mg/300 | mg |
| Vitamin B6 | 1.6 mg/300 | mg |
| Folic acid | 30.0 μg/300 | mg |
| Vitamin B12 | 2.4 μg/300 | mg |
| Pantothenic acid | 5 mg/300 | mg |
| L-ascorbic acid | 100 mg/300 | mg |

Since the above embodiments are described only for examples, the present invention is not limited to the above embodiments and various modifications or alterations can be easily made therefrom by those skilled in the art without departing from the scope of the present invention.

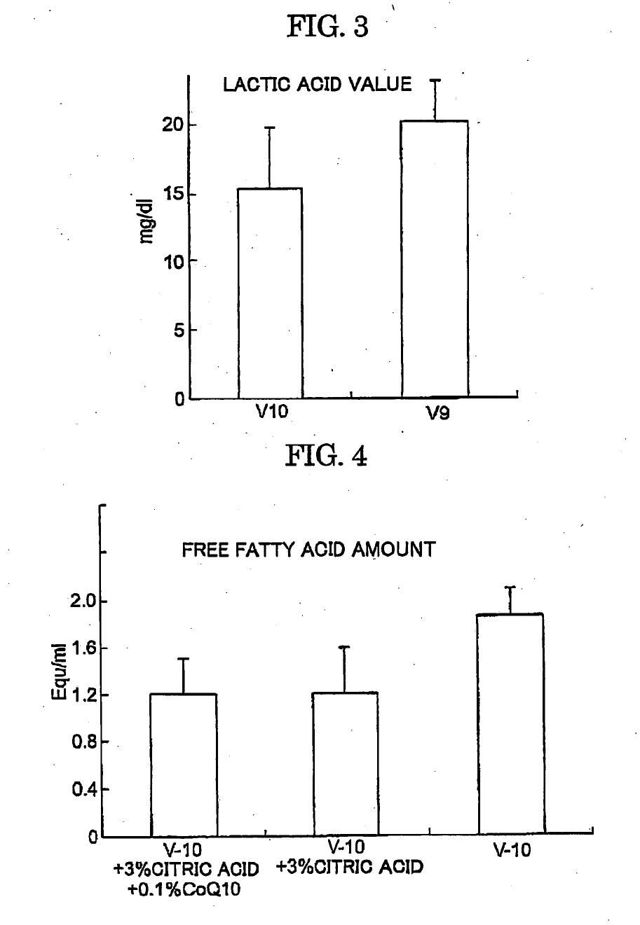

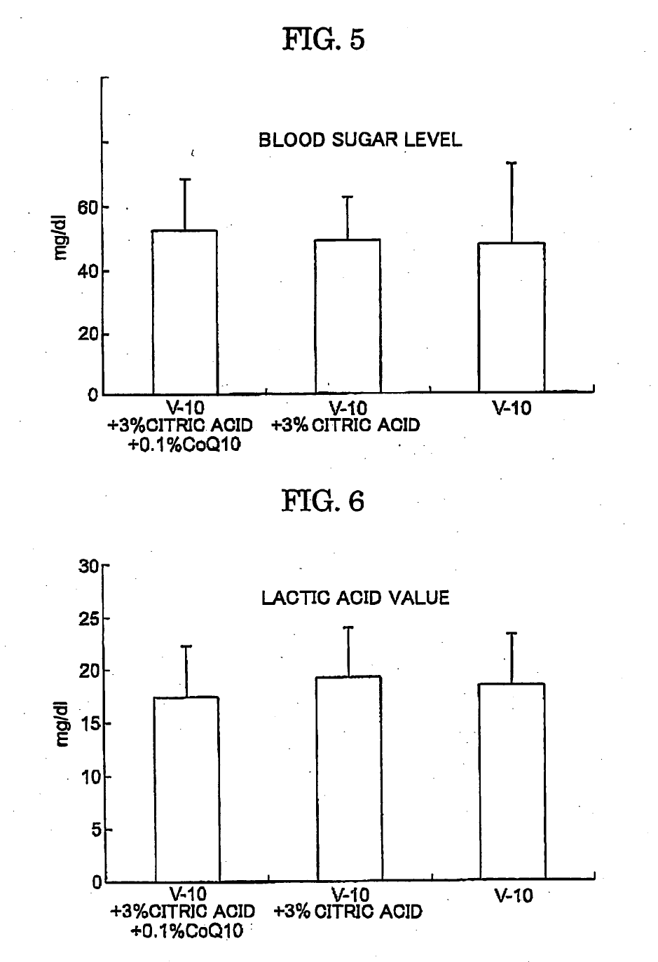

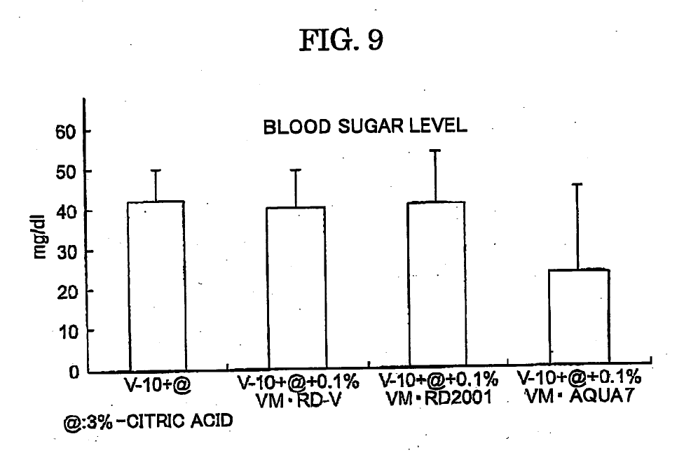

### BRIEF DESCRIPTION OF DRAWINGS

[FIG. 1] A graph showing blood sugar levels in blood in Example 1 and Comparative Example 1.

[FIG. 2] A graph showing lactic acid values in blood in Example 1 and Comparative Example 1.

[FIG. 3] A graph showing free fatty acid amounts in blood in Example 1 and Comparative Example 1.

[FIG. 4] A graph showing blood sugar levels in blood in Example 2.

[FIG. 5] A graph showing lactic acid values in blood in Example 2.

[FIG. 6] A graph showing free fatty acid amounts in blood in Example 2.

[FIG. 7] A graph showing blood sugar levels in blood in Example 3.

[FIG. 8] A graph showing lactic acid values in blood in Example 3.

[FIG. 9] A graph showing free fatty acid amounts in blood in Example 3.

1. Energy-imparting amino acid composition or amino acid solution containing glutamine comprising proline, alanine, valine, isoleucine, lysine and the glutamine.

2. The energy-imparting amino acid composition or amino acid solution as claimed in claim 1, wherein the respective amino acids are contained in such ratios as 4 to 30 moles of the proline, 0.1 to 12 moles of the alanine,4 to 8 moles of the valine, 3 to 9 moles of the isoleucine, 5 to 11 moles of the lysine and 0.1 to 4 moles of the glutamine.

3. Energy-imparting amino acid composition or amino acid solution containing glutamine comprising proline, alanine, valine, isoleucine, lysine, the glutamine and citric acid.

4. The engery-imparting amino acid composition or amino acid solution as claimed in claim 3, wherein the respective amino acids are contained in such ratios 4 to 30 moles of the proline 0.1 to 12 moles of the alaine, 4 to 8 moles of valine, 3 to 9 moles of the isoleucine, 5 to 11 moles of the lysine, 0.1 to 4 moles of the glutamine and 5 to 50 moles of the citric acid.

5. The energy-imparting amino acid composition or amino acid solution as claimed in claim 1, further comprising a glutamine-stabilizing sugar.

6. The energy-imparting amino acid composition or amino acid solution as claimed in claim 1, further comprising at least one COQ10 and vitamin-mix RD-V.

7. Energy-imparting amino acid composition or amino acid solution containing glutamine comprising a plurality of essential amino acids, the glutamine and glutamine-stabilizing sugar.

8. The energy-imparting amino acid composition or amino acid solution as claimed in claim 3, further comprising a glutamine-stabilizing sugar.

9. The energy-imparting amino acid composition or amino acid solution as claimed in claim 5, further comprising at least one COQ10 and vitamin-mix RD-V.

10. The energy-imparting amino acid composition or amino acid solution as claimed in claim 3, further comprising at least one COQ10 and vitamin-mix RD-V.
